(19) United States
(12) Patent Application Publication
(10) Pub. No.: US 2008/0108861 A1
(43) Pub. Date: May 8, 2008

(54) TREATMENT FOR FEMALE INCONTINENCE

(76) Inventors: Tricia Harris, Montague, MI (US); Tammie DeRochey, Montague, MI (US)

Correspondence Address: JENNINGS, STROUSS & SALMON, P.L.C., 201 E. WASHINGTON ST., 11TH FLOOR, PHOENIX, AZ, 85004, US

(21) Appl. No.: 11/934,128

(22) Filed: Nov. 2, 2007

Related U.S. Application Data
(60) Provisional application No. 60/856,551, filed on Nov. 2, 2006.

(51) Int. Cl. A61F 2/00

(52) U.S. Cl. 600/29

(57) ABSTRACT

The present invention provides for a device and method for the treatment of female urinary incontinence (UI). The disclosed device has a unique and novel shape that supports the urethra and absorbs any leakage. The device is placed through the female labia into the lower part of the female vagina to provide the urethral support and decrease the occurrence of female incontinence.

### CLAIM TO DOMESTIC PRIORITY

This application claims the benefit of priority of provisional application Ser. No. 60/856,551 filed on Nov. 2, 2006.

### FIELD OF THE INVENTION

This invention relates in general to the field of female incontinence and, more specifically, to a novel device and method for the treatment of female incontinence.

### BACKGROUND OF THE INVENTION

Over 54 million women suffer loss of bladder control also known as urinary incontinence (UI). These statistics are staggering when you consider the amount of time, money and inconvenience involved with traditional treatments. There are many degrees of UI. Some women may lose a few drops of urine while running, coughing or laughing, while others may have a debilitating degree of UI. UI is a medical problem often solved with surgery, medication, exercises, electric shock, diapers and pads. These are the most common solutions currently available for treating UI.

For decades UI has been associated with shame, embarrassment and silence. A true number of female suffers is not known because the problem remains under reported. The condition impacts quality of life and is associated with an increased number of falls, urinary tract infections and skin breakdown.

### BRIEF DESCRIPTION OF THE FIGURES

FIG. 1 is an illustration of a female urinary incontinence treatment device according to one embodiment of the present disclosure, with perspective view shown as FIG. 1A and side view shown as FIG. 1B.

FIG. 2 is an illustration of a female urinary incontinence treatment device according to an alternate embodiment of the present disclosure, with perspective view showing as FIG. 2A and side view shown as FIG. 2B.

FIG. 3 is a top view of the female urinary incontinence treatment device.

FIG. 4 is a bottom view of the female urinary incontinence treatment device.

FIG. 5 is a side view of a further embodiment of the female urinary incontinence treatment device.

FIG. 6 is an illustration of a female urinary incontinence treatment method according to the present disclosure.

### DETAILED DESCRIPTION OF VARIOUS EMBODIMENTS

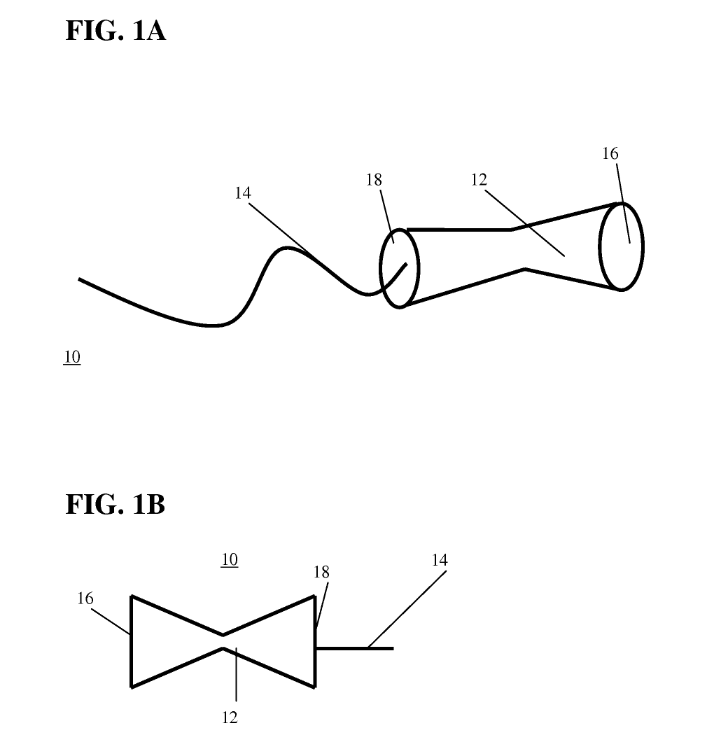

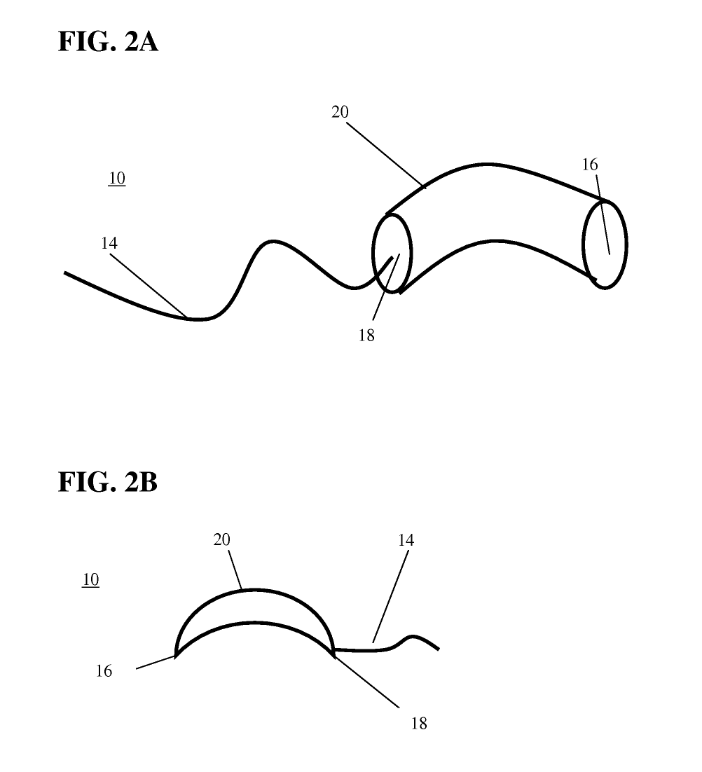

The present invention provides for a novel device and treatment for urinary incontinence in females. As shown in FIG. 1, one embodiment of the presently disclosed female incontinence device 10 comprises an hour-glass shaped body 12, with a circular insertion end 16 and a circular withdrawal end 18. As shown in FIG. 2, an alternate embodiment of the presently disclosed device comprises a curved or crescent-shaped body 20, also described herein as an arcuate cylindrical body, with circular insertion end 16 and circular withdrawal end 18.

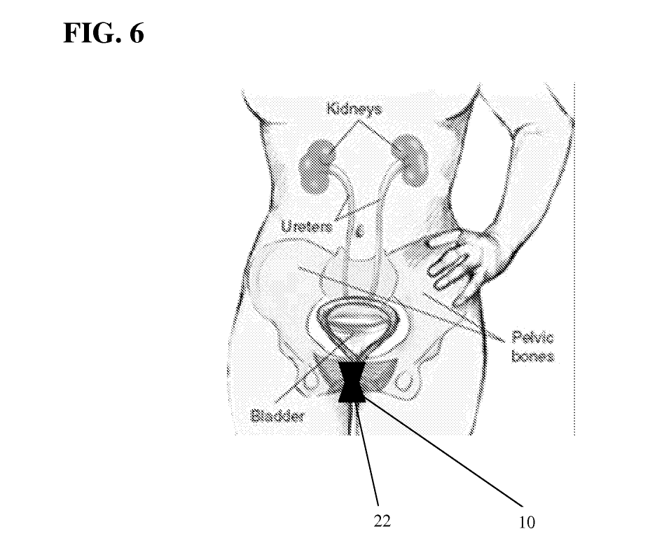

The unique shape of the disclosed device as shown in FIG. 1 and FIG. 2 is designed to support the urethra as well as absorb any leakage. A weakened pelvic floor cannot hold the urethra in its correct position, so any movement that puts pressure on the bladder may cause the urethra to lose its seal and allow urine to escape. The disclosed device replaces that support for the urethra and facilitates sealing of the urethra. Medical research has shown that support of the urethra is beneficial in relieving many women of UI problems. Specifically, device 10 consists of either an hour-glass shaped body 12 or crescent-shaped body 20 that is capable of being inserted through the labia of a female into the lower part of the vagina. FIG. 6 illustrates device 10 in use after being inserted through the female labia 22.

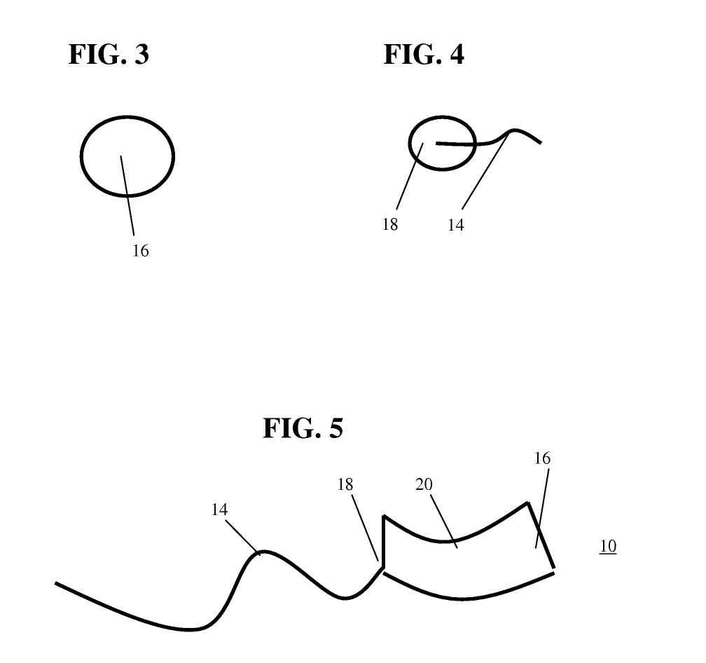

FIG. 3 specifically shows the top of device 10, namely insertion end 16. FIG. 4 specifically shows the bottom of the device, namely withdrawal end 18. Withdrawal end 18 also includes removal string 14, shown also in FIGS. 1 and 2, which allows the female to remove device 10 when it needs to be replaced or removed.

Device 10 is constructed of any material known in the art that is suitable for insertion into the female vagina. Preferably, device 10 a mass of absorbent material formed into a self-sustaining shape comprising an insertion end 16, a withdrawal end 18, and a center region, as shown in FIGS. 1 and 2.

As shown in FIG. 1, the center region of hour-glass shaped body 16 has at least one diameter which is less than the larger insertion end diameter and is less than a smaller withdrawal end diameter giving it an hour-glass like shape. The device is pinched-in approximately in the center of the body of the device, but the two sides of the device on either side of the pinched-in region need not be symmetrical. As noted, the withdrawal end diameter is smaller than the insertion end diameter, but both are larger than a diameter along body 12. However, the diameters of the insertion end and the withdrawal end may also be approximately equal, as shown in FIG. 1B.

In an alternate embodiment, as shown in FIG. 2, device 10 comprises an arcutuate cylindrical body, also described as an approximately crescent-shaped or curved body 20, also with insertion end 16 and withdrawal end 18 including withdrawal string 14. Crescent-shaped body 20 also has a larger diameter at insertion end 16 and is curved to a smaller diameter at withdrawal end 18. However, the diameter along the body may also be substantially uniform with the diameters of the insertion end and the withdrawal end also being approximately equal to each other and/or to the diameter of the body, as shown in FIG. 2B.

Both hour-glass shaped body 12 and crescent (or curved) shaped body 20 allow device 10 to effectively support the urethra in such a manner to relieve female urinary incontinence. The unique and novel shape of the device also allows for absorption of any residual leakage.

Device 10 can range in length from approximately four centimeters to approximately ten centimeters, with the insertion and withdrawal ends ranging from approximately one to three centimeters. The insertion and withdrawal ends may be the same diameter, or in other embodiments, the insertion end may have a greater diameter than the withdrawal end. Further, device 10 can either be inserted using an applicator-type device or can be inserted manually without an applicator.

The present device and method of using solves the short-term as well as the long-term UI problems with women. Most women will be comfortable using the disclosed device as it is similar in overall size and method of use to that of a tampon. They can be used at any time, in any situation, as often as needed, without inconvenience or embarrassment. The device is then disposed of after use, or changed as needed, preferably every four to eight hours.

This is in sharp contrast to surgeries to support the urethra which on average only offer relief for approximately 4 years. A patient would then need to have another surgery or seek additional treatment for long-term relief. The present device can be used over the long term, disposed of and replaced approximately every four to eight hours as needed.

Medications may often give long-term solutions but there is always risk of side effects or adverse reactions. Also, one must be very cautious if already taking medication for a different ailment. In comparison, there is very little risk of complications when using the disclosed device. The disclosed device offers immediate, pain-free relief, giving many women the option of being in control of their bodies and the ability to attend to their needs on a very personal level.

Further, because this device can be used only when needed and is not a drug, pregnant women can use the device during pregnancy, on an as needed basis without worrying about the effects of drugs or other procedures on the fetus.

The demand for natural relief, free from pharmaceutical treatment, is evident in society with an increase in popularity and use of holistic, naturopathic, homeopathic and various other medical solutions. These are very popular alternatives to traditional medical practices. The disclosed device offers treatment with a solution as pure and natural as any woman could find. No complicated surgeries, no urethra splints, no medication or electric shock are required. Giving women a sense of empowerment and control over UI, in a natural way, will offer millions of women a new sense of independence and confidence.

Because UI is a personal medical situation, it requires a personal solution, with affordable relief, not only from UI but relief from the fear of having to choose between currently-available, expensive and often inconvenient choices. Most of these options require decisions which may affect patients for the rest of their lives.

Device 10 is inexpensive, easy to carry, easy to use and convenient. When the cost is compared to all other treatments, it is a fraction of the cost a patient would expect to pay for relief. Considering, approximately 54 million women suffer from some form of UI, the disclosed device and method of treatment could easily be accessible and affordable to nearly all these women. Device 10 is a simple inexpensive solution that will help treat a common, female ailment with ease, cost effectiveness, confidence and practicality.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of a preferred embodiment and best mode of the invention known to the applicant at this time of filing the application has been presented and is intended for the purposes of illustration and description. It is not intended to be exhaustive nor limit the invention to the precise form disclosed and many modifications and variations are possible in the light of the above teachings. The embodiments were chosen and described in order to best explain the principles of the invention and its practical application and to enable others skilled in the art to best utilize the invention in various embodiments and with various modifications as are suited to the particular use contemplated. Therefore, it is intended that the invention not be limited to the particular embodiments disclosed for carrying out the invention.

What is claimed is:

1. A device for the treatment of female incontinence, comprising:
an arcuate cylindrical body of uniform diameter, said body having a withdrawal end, an insertion end and a center region, wherein the insertion end is suitable for insertion through the female labia, and is opposed to the withdrawal end, and the center region is located between the insertion end and the withdrawal end; the center region having a diameter which is substantially uniform along the length of the body, and said center region diameter being approximately equal to the insertion end diameter and the withdrawal end diameter; and a removal string coupled to the withdrawal end.

2. The device of claim 1, wherein the body is comprised of an absorbent material.

3. The device of claim 1, wherein the body is comprised of a material suitable for insertion into the female vagina.

4. The device of claim 1, wherein the body is capable of providing urethral support.

5. The device of claim 1, wherein the body is comprised of rayon.

6. The device of claim 1, wherein the body is comprised of cotton.

7. A method for treating female incontinence, comprising:
inserting into the female vagina, a device, said device including an arcuate cylindrical body of uniform diameter, said body having a withdrawal end, an insertion end and a center region, wherein the insertion end is suitable for insertion through the female labia, and is opposed to the withdrawal end, and the center region is located between the insertion end and the withdrawal end; the center region having a diameter which is substantially uniform along the length of the body, and said center region diameter being approximately equal to the insertion end diameter and the withdrawal end diameter; and a removal string coupled to the withdrawal end, wherein female incontinence is reduced.

8. The method of claim 7, further comprising removing the device from the female vagina approximately four to eight hours after insertion.

9. The method of claim 8, further including inserting a second device into the female vagina.

10. The method of claim 7, wherein the body is comprised of an absorbent material.

11. The method of claim 7, wherein the body is comprised of a material suitable for insertion into the female vagina.

12. The method of claim 7, wherein the body is capable of providing urethral support.

13. The method of claim 7, wherein the body is comprised of rayon.

14. The method of claim 7, wherein the body is comprised of cotton.

15. A device for the treatment of female incontinence, comprising:
an hour-glass shaped body, said body a having a withdrawal end, an insertion end and a center region, wherein the insertion end is suitable for insertion through the female labia, and is opposed to the withdrawal end, and the center region is located between the insertion end and the withdrawal end; the center region having a diameter which is less than both the insertion end the withdrawal end diameters, which are approximately equal; and a removal string coupled to the withdrawal end.

16. The device of claim 15, wherein the body is comprised of an absorbent material.

17. The device of claim 15, wherein the body is comprised of a material suitable for insertion into the female vagina.

18. The device of claim 15, wherein the body is capable of providing urethral support.

19. The device of claim 15, wherein the body is comprised of rayon.

20. The device of claim 15, wherein the body is comprised of cotton.

21. A device for the treatment of female incontinence, comprising:
an arcuate cylindrical body of variable diameter, said body having a withdrawal end, an insertion end and a center region, wherein the insertion end is suitable for insertion through the female labia, and is opposed to the withdrawal end and the center region is located between the insertion end and the withdrawal end; the center region having a diameter which is less than both the insertion end diameter and the withdrawal end diameter; and a removal string coupled to the withdrawal end.

22. The device of claim 21, wherein the insertion end diameter and the withdrawal end diameter are approximately equal.

23. The device of claim 21, wherein the insertion end diameter is greater than the withdrawal end diameter.

24. A device for the treatment of female incontinence, comprising:
an hour-glass shaped body, said body a having a withdrawal end, an insertion end and a center region of variable diameter which is less than both the insertion end diameter and the withdrawal end diameter, wherein the insertion end is suitable for insertion through the female labia, and is opposed to the withdrawal end, and the center region is located between the insertion end and the withdrawal end; the center region having its smallest diameter approximately equidistant between the insertion end and the withdrawal end; and a removal string coupled to the withdrawal end.

25. The device of claim 24, wherein the insertion end diameter is greater than the withdrawal end diameter.
